(19) United States
(12) Patent Application Publication
(10) Pub. No.: US 2020/0100701 A1
(43) Pub. Date: Apr. 2, 2020

(54) VASCULAR ACCESS DEVICE AND METHOD FOR TRACKING A MEDICAL INSTRUMENT IN A BODY REGION OF A PATIENT

(71) Applicant: Siemens Healthcare GmbH, Erlangen (DE)

(72) Inventors: Arne Hengerer, Moehrendorf (DE); Volker Matschl, Bamberg (DE)

(73) Assignee: Siemens Healthcare GmbH, Erlangen (DE)

(21) Appl. No.: 16/584,687

(22) Filed: Sep. 26, 2019

(30) Foreign Application Priority Data
Sep. 27, 2018 (EP) 18197218.3

(51) Int. Cl. A61B 5/06; A61B 5/055; A61M 39/02; G01R 33/28

(52) U.S. Cl. CPC A61B 5/065; A61B 5/055; A61M 39/0247; A61M 2039/0261; G01R 33/287; A61M 2039/0258; A61M 2039/0273; A61B 5/066

(57) ABSTRACT

The disclosure relates to a vascular access device, comprising a vascular cannula having a catheter portion for insertion into a blood vessel such as a vein of a patient, and a fixing portion for fixing the vascular cannula to a skin of a patient. The fixing portion and/or a plaster used to fix the fixing portion to the skin comprises at least one local reception coil element for receiving magnetic resonance signals.

### CROSS-REFERENCE TO RELATED APPLICATIONS

The present application claims the benefit of the filing date of European patent application no. EP18197218.3, filed on Sep. 27, 2018, the contents of which are incorporated herein by reference in their entirety.

### TECHNICAL FIELD

The disclosure relates to a vascular access device including a vascular cannula having a catheter portion for insertion into a blood vessel (e.g. a vein) of a patient and a fixing portion for fixing the vascular cannula to a skin of a patient. The disclosure further relates to a method for tracking a medical instrument in a body region of a patient.

### BACKGROUND

In medicine, for many therapies or examinations, access to a blood vessel of a patient, in most cases a vein, is required. In this regard, vascular access devices (VAD), sometimes also called venous access devices, are used. Such vascular access devices usually comprise a vascular cannula having a catheter portion that is inserted into the blood vessel. The vascular cannula also usually has a fixing portion, in particular comprising wings, which enable the vascular cannula to be fixed to the skin of the patient. To this end, adhesives and/or plasters may be used.

The vascular cannula may further comprise a valve to allow injection of medications, for example drugs, using a syringe and/or an end which allows connection to an intravenous infusion line. Often, a so-called “mandrin” is additionally provided, which is removed after successful positioning of the vascular cannula. Mandrins are usually made of metal and positioned on the inside of the vascular cannula, in particular the catheter portion. Conventional solutions with regards to the guidance and tracking of vascular access devices in magnetic resonance-guided interventions and/or procedures are inadequate.

### SUMMARY

To place a VAD, an insertion region is usually first treated with a local skin antiseptic. After this, the insertion region may be covered using a surgical incise drape. Then, the actual insertion takes place using a local anesthetic. The blood vessel is typically punctured using the vascular cannula. The insertion into the blood vessel can be seen from blood fill that occurs in the back of the cannula.

While the so-created vascular access may be used for medication, it is often also used as an entry point for a medical instrument. For example, the entry point may be utilized for a guide wire for another instrument, such as a therapeutic catheter or the like. For instance, after the mandrin is removed, the medical instrument is inserted into the blood vessel via the vascular cannula, which is often done using an image modality for tracking angiography, for example. In the case of such a minimally invasive intervention, if the medical instrument is a guide wire, the guide wire can be fixed and the vascular cannula removed. While doing so, the blood vessel is compressed at the access point to fix the position of the guide wire. Afterwards, a sluice or the therapeutic or treatment catheter is slid over the guide wire to its target position. The guide wire can then subsequently be removed.

For such minimally invasive interventions on a patient, for which often the vena cava is used as vascular access point, central venous catheters are often used. In the case that a peripheral vein is used to establish access, a peripheral venous catheter may be used. For tracking the insertion of the vascular cannula (or later on the insertion of the medical instrument through the venous cannula, as already mentioned), the progress may be tracked using an imaging modality, for example x-ray angiography.

However, if an intervention is to be guided by magnetic resonance as the imaging modality, in most cases magnetic resonance imaging does not provide enough local information to allow using this mobility for tracking at the access site. In this case, the venous access can be established and the medical instrument can be introduced without image guidance. It has been proposed to add a second imaging modality, for example ultrasound imaging or x-ray imaging.

It is an object of the current disclosure to provide a vascular access device and a method using this device allowing better guidance and tracking in magnetic resonance-guided interventions and/or procedures. This object is achieved by providing a vascular access device and a method according to the advantageous embodiments as described in the claims and elsewhere throughout the disclosure.

### BRIEF DESCRIPTION OF THE DRAWINGS/FIGURES

The accompanying drawings, which are incorporated herein and form a part of the specification, illustrate the embodiments of the present disclosure and, together with the description, further serve to explain the principles of the embodiments and to enable a person skilled in the pertinent art to make and use the embodiments.

Further features, details, and advantages of the present disclosure arise from the following description of preferred exemplary embodiments and also on the basis of the drawings, in which:

FIG. 1 is a schematic drawing of an example vascular access device (VAD), according to an embodiment of the present disclosure;

FIG. 2 is an equivalent circuit diagram of a first embodiment of a local reception coil element, according to an embodiment of the present disclosure; and

FIG. 3 is an equivalent circuit diagram of a second embodiment of a local reception coil element, according to an embodiment of the present disclosure.

The exemplary embodiments of the present disclosure will be described with reference to the accompanying drawings. The drawing in which an element first appears is typically indicated by the leftmost digit(s) in the corresponding reference number.

### DETAILED DESCRIPTION

According to the disclosure, in a vascular access device as described, the fixing portion and/or a plaster used to fix the fixing portion to the skin comprises at least one local reception coil element for receiving magnetic resonance signals during placement of the vascular cannula and/or during placement of an instrument through the cannula.

In an embodiment, a local reception coil element is thus provided on the fixing portion. The fixing portion may comprise at least one wing, on or integrated into which the local reception coil element is positioned. Alternatively, a plaster with which the wing is to be fixed to the skin of the patient may have the local reception coil element attached to it or integrated into it.

Generally, the local reception coil element may be added in an inexpensive way. For instance, the local reception coil element may be printed onto the plaster and/or the fixing portion, in particular the wing, or the local reception coil element may be provided on a flexible circuit board attached to the plaster and/or the fixing portion, in particular the wing. In this respect, the local reception coil element, (e.g. the whole VAD), may be disposable and/or configured for one-time use. This means that the vascular cannula, the plaster, and/or its local reception coil element does not have to be sterilized. Again, since the local reception coil element can be produced very cost-effective, a one-time use is feasible from a financial point of view. As is known, the vascular cannula may be made from any suitable materials, such as polymers (e.g. plastic) or the like.

In summary, while the VAD and the radio frequency coils are, according to the state of the art, two independent systems, a local coil is usually positioned first, whereafter sterility at the access site is ensured and vascular access is established. The current embodiments of the present disclosure, however, advantageously combine the VAD and the local reception coil.

In an embodiment, two steps in the workflow are combined, namely placement of the local coil and establishing the vascular access. Additionally, an optimal position of the access site and the reception coil is ensured. In particular, if the local reception coil element is fixed to the fixing portion, accidental movement of the local reception coil element is prevented.

In this case, a low-cost local reception coil element suffices to allow adequate image quality for the specific application. In particular, if the local reception coil element is configured for one-time use, the local reception coil can be understood as an expendable item, which can be distributed using existing distribution channels (e.g. the VAD industry).

By providing the local reception coil as a part of the VAD, the local reception coil element is placed exactly where it needs to be to allow better image quality, such as better signal-to-noise-ratio (SNR), in an imaging area covering the penetration depth and the penetrated blood vessel (e.g. a vein) for magnetic resonance imaging. That is, the local reception coil element, which is a local reception coil element for magnetic resonance imaging, is configured to receive magnetic resonance signals from a body region comprising the access site and the vessel into which the vascular cannula is inserted, and to directly or indirectly transfer these received magnetic resonance signals to a magnetic resonance imaging device in which the patient is placed, such as for an intervention or other procedure requiring vascular access. This may occur, for instance, during a magnetic resonance imaging, or scanning, operation performed via the magnetic resonance imaging device.

In the scope of the current disclosure, it is in principle conceivable that the VAD further comprises at least one connecting device having a cable, for the local reception coil element and/or that the local reception coil element comprises a pre-amplifier and/or at least one adaptation and/or detuning circuit. The at least one local reception coil element may be a conventional magnetic resonance local coil having for example a detuning circuit and/or an adaptation circuit and/or a pre-amplifier, such that control and signal transmission may take place via a connecting device, such as a coil plug. It may also be possible to use the local reception coil element for transmitting magnetic resonance signals regarding excitation. However, such a configuration may be, in some instances, less preferred with regards to the cost and optional sterilization.

In a preferred embodiment, the local reception coil element is configured to inductively couple to an external reception coil, such as a body coil and/or an external local coil of a magnetic resonance imaging device. In this way, a cable-free local reception coil element is provided. To do so, the local reception coil element is inductively coupled to a body coil of the magnetic resonance imaging device and/or a local coil of the magnetic resonance imaging device.

To achieve the inductive coupling to the external reception coil, the local reception coil element may comprise a self-resonant conducting structure and/or circuit. That is, a self-resonant structure can be provided on the substrate of the local reception coil element, or, alternatively, a self-resonant circuit can be formed using discrete electronic components, for example surface mount (SMD) capacitors. Self-resonance can, for example, be achieved by providing overlapping conductor areas, which couple capacitively. The conductors themselves act as inductances.

When using a self-resonant local reception coil element, the local reception coil element may be detuned during transmission of the excitation signals by corresponding transmitter coils of the magnetic resonance imaging device, that is, during excitation of spins to be imaged using a high-frequency field (B1 field). Thus, in an embodiment, the local reception coil element further comprises at least one detuning element for detuning the local reception coil element during a magnetic resonance transmitting phase. In this manner, an excessive increase of the B1 field, and thus an endangerment of the patient, are prevented.

In embodiments, at least one detuning element may comprise at least one switching element, such as a diode, for example. The switching element may close upon a predefined voltage being induced into the local reception coil element. The diodes may be any suitable type, such as PIN diodes for instance. In embodiments, two anti-parallel diodes may be used such that either one of the diodes allows current to pass through an additional detuning capacitor or inductance, or both diodes allow current to pass and thus “intrinsically decouple” two then-formed sub-loops of the local reception coil element's main loop. Two anti-parallel PIN diodes may, for example, be used in the middle of the resonance loop of the local reception coil element, such that the two anti-parallel PIN diodes neutralize the loop in a global view, which is referred to as intrinsic decoupling. In alternative embodiments, at least one PIN diode may allow adding or removing additional capacitances or inductances of the local reception coil element. PIN diodes allow current to pass once a certain voltage is applied, meaning, in this case, that a voltage surpassing such a switching voltage is induced into the local reception coil element by the magnetic resonance excitation field (B1 field). When receiving, however, induced voltages are low and the at least one diode does not allow current to pass.

The local reception coil element may have any suitable shape and/or size. In embodiments, the local reception coil element may have a maximum dimension of 2 to 5 cm. Local reception coil elements of this size inductively couple to external reception coils, such as a body coil of the magnetic resonance imaging device, for example. Additionally, the depth of penetration of the local reception coil element, i.e. the extension of the imaging region into the patient, corresponds essentially to the diameter of the local reception coil element.

Of course, the VAD may have additional, less, or alternate components, as known from the state of the art. In particular, the VAD may further comprise a mandrin and/or a protective cap.

In the case of a mandrin, it may be preferred if the mandrin comprises a wing having an additional local reception coil element. For example, such an additional local coil reception element may be used to obtain magnetic resonance signals during placement of the vascular access device, while the mandrin is still in place inside the vascular cannula (e.g. in the catheter portion).

It is additionally noted that inductively coupling the local reception coil element to an external reception coil usually only reduces the SNR by about 50%, whereas using the local reception coil element, compared to using only a body coil, increases the SNR by a factor of about 5, so that using the inductive coupling is acceptable.

The disclosure also comprises a method for tracking a medical instrument in a body region of a patient, the medical instrument being inserted through a vascular cannula of a VAD according to the disclosure, wherein magnetic resonance signals are received by the local reception coil element and used by a magnetic resonance imaging device to reconstruct an image of the body region. All features and comments regarding the VAD may correspondingly be applied to the method according to the disclosure, as further described herein.

In particular, the local reception coil element may be inductively coupled to an external reception coil of the magnetic resonance imaging device, such that the location of the local reception coil element appears “bright” in the magnetic resonance data of the external reception coil. Enlarging this “brighter” image area yields high quality image data of an imaging region of the local reception coil element.

In embodiments, the body region from which magnetic resonance signals are received by the local reception coil element, i.e. the imaging region of the local reception coil element, and from which a corresponding magnetic resonance image may be reconstructed, may have any suitable size and/or shape. For instance, the body region may have at least the length of a mandrin of the VAD plus 5 mm parallel to a projection of the mandrin onto the skin surface and/or at least a length of 25 mm perpendicular thereto along the skin surface, and/or at least the penetration depth of the mandrin plus 15 mm perpendicular to the skin surface. In this manner, for example, the correct insertion of a guide wire may be tracked.

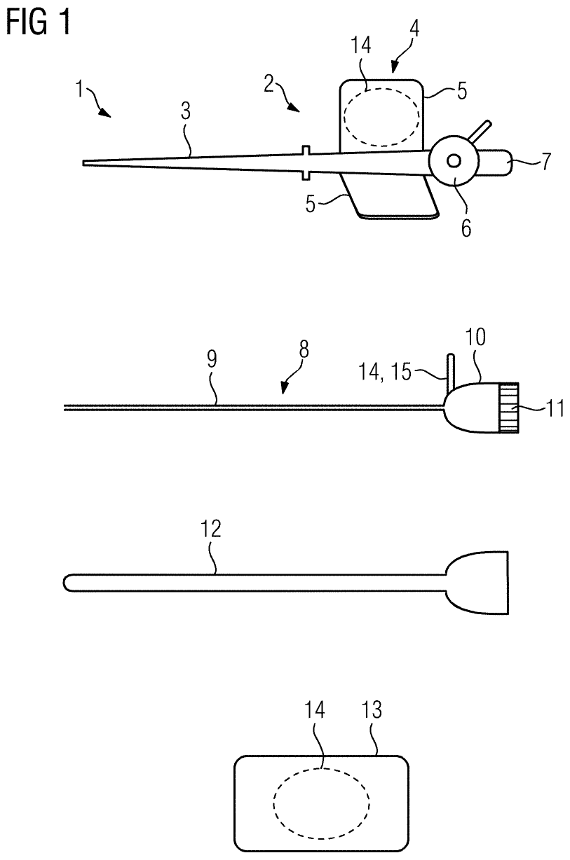

FIG. 1 is a schematic drawing of an example vascular access device (VAD), according to an embodiment of the present disclosure. As an essential component, the VAD 1 comprises a vascular cannula 2, which has a catheter portion 3 whose tip is inserted into a blood vessel (e.g. a vein), and a fixing portion 4, in this case having two wings 5, for fixing the vascular cannula 2 onto the skin of a patient as soon as the catheter portion 3 is in place. The number of wings 5 is by way of example and not limitation, and the VAD 1 may include any suitable number of wings depending upon the desired application. The vascular cannula 2 may additionally comprise a valve 6 to allow injection of medication with a syringe, and an end 7 to which an intravascular infusion line can be connected and/or through which a medical instrument, for example a guide wire for a therapeutic or treatment catheter, may be inserted into the vascular cannula 2 and therefore the blood vessel.

The VAD 1 also comprises a mandrin 8 having a hollow steel portion 9, a blood container 10 and a cap 11. Usually, the mandrin 8 is inserted in the vascular cannula 2 such that the front end of the hollow steel portion 9 is used as a needle to penetrate the skin of a patient and find the suitable blood vessel, wherein insertion into the blood vessel is indicated by blood filling the blood container 10.

The VAD 1 may additionally comprise a protective cap 12 and/or a plaster 13 for fixing a wing 5 to the skin of a patient after the vascular cannula 2 and in particular the catheter portion 3 is in place.

According to an embodiment of the present disclosure, at least one wing 5 of the vascular cannula 2 and/or the plaster 13 may be provided with at least one local reception coil element 14 for magnetic resonance imaging. It is noted that the mandrin 8 may also have a wing 15, which may have a local reception coil element 14 attached to it or integrated into it.

It is noted at this point that the vascular cannula 2, the blood container 10, the wing 15, and the cap 11, as well as the protective cap 12 are, in this embodiment, all made of plastic, but may be made of any suitable type of polymer or other material, which may be the same or different than one another.

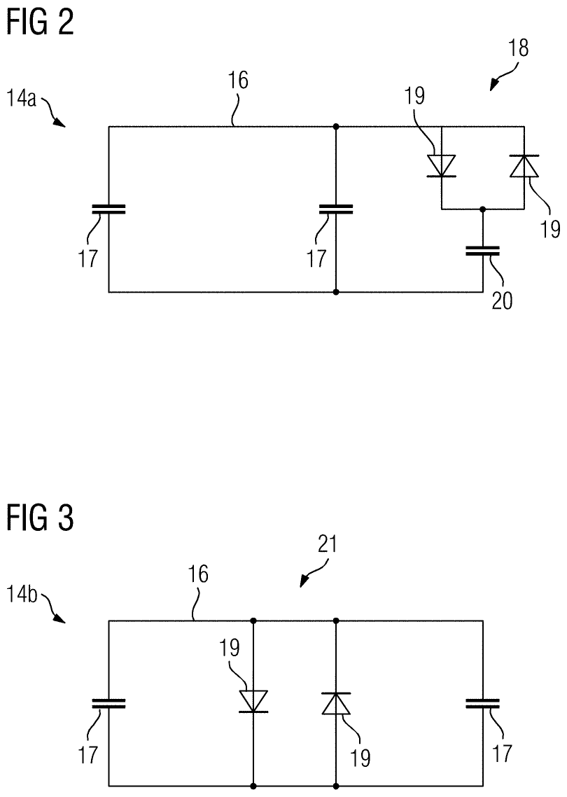

The local reception coil element 14 may, in an embodiment, resemble a local coil, having a pre-amplifier, a detuning circuit and/or an adaptation circuit. In such a case, the vascular cannula 2 may have a connecting device with a cable for the local reception coil element. It is, however, preferred and realized in the shown embodiments that the at least one local reception coil element 14 is inductively coupled to an external reception coil of a used magnetic resonance imaging device, as further explained below with respect to FIGS. 2 and 3.

The local reception coil element 14 may be implemented in accordance with any suitable type of circuit materials. For instance, while it is conceivable to provide the local reception coil element 14 on a circuit board (e.g. a flexible circuit board) the reception coil element 14 may alternatively be printed or integrated (e.g. via molding) the local reception coil element 14 directly onto or into, respectively, the wing 5 and/or the wing 15, and/or the plaster 13, wherein some embodiments include the local reception coil element 14 being implemented in at least one of the wings 5.

As the penetration depths from which magnetic resonance signals may be received by the local reception coil element 14 essentially correspond to its dimensions, a maximum dimension (e.g. a diameter of the loop of the local reception coil element 14) may be, for instance, between 2 cm-5 cm. For example, the local reception coil element 14 may include a loop having a diameter between 2 cm-5 cm, less than 2 cm, less than 5 cm, etc. Such dimensions also allow inductive coupling to the external reception coil.

To enable inductive coupling to the external reception coil of a magnetic resonance imaging device, e.g. a local coil and/or a body coil, the local reception coil element 14 comprises a self-resonant structure and/or circuit. In the case of a self-resonant conductor structure, overlapping conductors provide capacitance, while the conductors themselves act as inductances. In a self-resonant circuit, discrete electronic components, for example SMD capacitors, can be used.

FIG. 2 is an equivalent circuit diagram of a first embodiment of a local reception coil element, according to an embodiment of the present disclosure. FIG. 2 shows an equivalent circuit diagram of a first embodiment of a local reception coil element 14a. The local reception coil element 14a comprises a primary loop 16 for receiving magnetic resonance signals, in this case realized as a self-resonant conductor structure such that only the capacitors 17 created by overlapping conductors are shown. Inductances, as explained, are created by the conductors themselves.

The local reception coil element 14a further comprises a detuning device 18 having two diodes 19 (e.g. PIN diodes) acting as switching elements, which are provided in an anti-parallel configuration. As a further detuning element, an additional capacitor 20 is provided.

As the diodes 19 allow current to pass once a certain voltage is surpassed, self-resonance during the transmitting phase of the magnetic resonance imaging device causes one of the diodes 19 to conduct, allowing current to pass such that the additional capacitor 20 is added and the main loop 16 is detuned, preventing excessive increase of the local B1 field. In other words, during the transmitting phase, the local reception coil element 14a is decoupled from the transmitting coil via the frequency shift. In the case of signal reception during the receiving phase of the magnetic resonance imaging device, the low induced voltages do not suffice to allow current to pass through the diodes 19 (i.e. neither of the diodes 19 conduct).

FIG. 3 is an equivalent circuit diagram of a second embodiment of a local reception coil element, according to an embodiment of the present disclosure. FIG. 3 shows an alternative embodiment of a local reception coil element 14b, in which the detuning device 21 again comprises two anti-parallel diodes 19 (e.g. PIN diodes) in the center of the loop 16. In this configuration, when the voltage limit is surpassed, the diodes 19 conduct and allow current to pass due to an applied B1 field in a transmitting phase of the magnetic resonance imaging device. In this scenario, the loop 16 is essentially divided into two sub-loops neutralizing the loop 16 globally by intrinsic decoupling.

The local reception coil element 14, 14a, 14b of the VAD 1 can be used to provide magnetic resonance image data of high SNR from an imaging region, which may for instance cover the whole access site. Such image data can be evaluated to assess the correct positioning of the vascular access device 1, to track, and/or to guide insertion of a medical instrument, such as a guide wire, into the patient using the VAD 1.

Although the embodiments of the present disclosure have been illustrated and described in detail using the preferred exemplary embodiment, the disclosure is not limited by the disclosed examples, and a person skilled in the art can derive other variations therefrom without departing from the scope of protection of the disclosure.

What is claimed is:

1. A vascular access device (VAD), comprising:
a vascular cannula including a catheter portion configured for insertion into a blood vessel of a patient; and
a fixing portion configured fix the vascular cannula to skin of the patient,
wherein at least one of the fixing portion and/or a plaster used to fix the fixing portion to the skin of the patient includes at least one local reception coil element configured to receive magnetic resonance signals from a magnetic resonance device during a magnetic resonance imaging operation.

2. The VAD according to claim 1, wherein the at least one local reception coil element is positioned on or integrated into the at least one wing included in the fixing portion.

3. The VAD according to claim 1, wherein the at least one local reception coil element is printed onto at least one of the plaster and/or the at least one wing included in the fixing portion.

4. The VAD according to claim 1, wherein the at least one local reception coil element is provided on a flexible circuit board that is attached to at least one of the plaster and/or the at least one wing included in the fixing portion.

5. The VAD according to claim 1, wherein the VAD is configured for one-time use.

6. The VAD according to claim 1, further comprising:
at least one connecting device having a cable coupled to the at least one local reception coil element.

7. The VAD according to claim 1, wherein the at least one local reception coil element includes at least one of a pre-amplifier, an adaptation circuit, and/or a detuning circuit.

8. The VAD according to claim 1, wherein the at least one local reception coil element is configured to inductively couple to at least one of an external body coil and/or an external local coil.

9. The VAD according to claim 1, wherein the at least one local reception coil element comprises a self-resonant conducting structure and/or circuit.

10. The VAD according to claim 1, wherein the at least one local reception coil element comprises at least one detuning element for detuning the at least one local reception coil element during a transmitting phase of the magnetic resonance imaging operation.

11. The VAD according to claim 10, wherein the at least one detuning element comprises at least one diode configured to conduct upon induction of a predefined voltage into the at least one local reception coil element.

12. The VAD according to claim 1, wherein the at least one local reception coil element includes a loop having a diameter that ranges between 2 cm and 5 cm.

13. The VAD according to claim 1, further comprising:
a mandrin.

14. The VAD according to claim 13, wherein the mandrin comprises a wing having an additional local reception coil element.

15. A method for tracking a medical instrument in a body region of a patient, the method comprising:
inserting the medical instrument through a vascular cannula of a vascular access device, the vascular cannula including a catheter portion for insertion of the medical instrument into a blood vessel of the patient;
fixing, via a fixing portion of the vascular access device, the vascular cannula to a skin of the patient;
receiving, via at least one the local reception coil element device during a magnetic resonance imaging operation, magnetic resonance signals used by a magnetic resonance imaging device to reconstruct an image of the body region of the patient; and
tracking, using the magnetic resonance signals, the inserting of the medical instrument through the vascular cannula of the vascular access device.

16. The method according to claim 15, wherein the body region from which the magnetic resonance signals are received by the at least one local reception coil element has at least a length of a mandrin of the vascular access device plus 5 mm parallel to a projection of the mandrin onto the skin surface of the patient.

17. The method according to claim 15, wherein the body region from which the magnetic resonance signals are received by the at least one local reception coil element has at least a length of 25 mm along the skin surface of the patient.

18. The method according to claim 15, wherein the body region from which the magnetic resonance signals are received by the at least one local reception coil element has a penetration depth of the mandrin plus 15 mm perpendicular to the skin surface of the patient.

19. The method according to claim 15, further comprising:
detuning, via a detuning element included in the at least one local reception coil element, at least one local reception coil element during a transmitting phase of the magnetic resonance imaging operation.

20. The method according to claim 19, wherein the act of detuning the at least one local reception coil element further comprises:
conducting, via at least one diode included in the at least one detuning element, upon induction of a predefined voltage into the at least one local reception coil element.
